(19) United States
(12) Patent Application Publication
(10) Pub. No.: US 2003/0070674 A1
(43) Pub. Date: Apr. 17, 2003

(54) Use of aerosolized compounds in the treatment of exercise induced pulmonary hemorrhage in an equine

(76) Inventors: Bryan Perry, West Seneca, NY (US); Christopher Miller, North Vancouver (CA); Douglas Hole, Edmonton (CA)

Correspondence Address: Kevin D. McCarthy, Roach, Brown, McCarthy & Gruber, P.C., 1620 Liberty Building, Buffalo, NY, 14202, US

(21) Appl. No.: 10/269,738

(22) Filed: Oct. 14, 2002

Related U.S. Application Data
(60) Provisional application No. 60/328,664, filed on Oct. 12, 2001.
(60) Provisional application No. 60/329,227, filed on Oct. 12, 2001.

(51) Int. Cl. A61M 11/00

(52) U.S. Cl. 128/200.14; 128/203.12

(57) ABSTRACT

The administration of aerosolized nitric oxide donors or type V phosphodiesterase inhibitor in controlled doses to horses during high intensity exercise is described. Aerosolized nitric oxide donors (in conjunction with intravenous type V phosphodiesterase inhibitor) and type V phosphodiesterase inhibitor are beneficial to prevent exercise induced pulmonary hemorrhage by reducing transmural pulmonary artery pressure with a concomitant decrease in pulmonary capillary stress failure. The administration of aerosolized nitric oxide donors and type V phosphodiesterase inhibitors is viewed as a novel therapeutic modality in the alleviation of capillary stress failure and EIPH in performance horses.

### CLAIM OF PRIOIRTY

This application claims priority to U.S. provisional patent applications serial Nos. 60/328,666 and 60/329,227, both of which were filed on Oct. 12, 2002.

### FIELD OF THE INVENTION

The present invention generally relates to the pulmonary hemodynamics of a horse (a.k.a. equine), and more particularly decreasing pulmonary hemorrhaging in the equine during exercise. In other words, the present invention relates to the decreased occurrence of exercise induced pulmonary hemorrhage (EIPH) in performance horses.

### BACKGROUND OF THE INVENTION

Although numerous hypotheses have been advanced in recent years, it is generally suggested that pulmonary capillary stress failure is a causal determinant of exercise induced pulmonary hemorrhage in performance animals such as horses. It has been known to those of skill in the art that increased high pulmonary artery pressure, pulmonary artery wedge pressure and stress failure at the capillary level within a horse are due to increased transmural pressure during strenuous exercise of the equine. It is further known that the horse has a relatively thin pulmonary blood-gas barrier to facilitate oxygen uptake during high intensity exercise. During exercise, pulmonary blood flow increases by as much as eight-fold to satisfy the increased oxygen need. The concurrent increase in pulmonary artery pressure is compensated for by a reduction in pulmonary vascular resistance and functional recruitment of the capillary bed.

The introduction of N-nitro-L-arginine methyl ester (L-NAME) directly into the pulmonary artery of a horse subjected to treadmill tests is known to synthesize nitric oxide (NO) within horses. The L-NAME compound has been shown to inhibit the in-situ production of NO which is known to regulate basal pulmonary vascular tone in many mammal species. During exercise, a reduced level of NO in the lungs results in a significant increase in the pulmonary artery pressure. In contrast, the introduction of L-arginine, a structural analogue of L-NAME, into the pulmonary artery of exercised horses reverses the restricted production of NO with a concomitant reduction in pulmonary artery pressure.

### SUMMARY OF INVENTION

The administration of aerosolized nitric oxide donors or type V phosphodiesterase inhibitor in controlled doses to horses during high intensity exercise is described. Aerosolized nitric oxide donors (in conjunction with intravenous type V phosphodiesterase inhibitor) and type V phosphodiesterase inhibitor are beneficial to prevent exercise induced pulmonary hemorrhage by reducing transmural pulmonary artery pressure with a concomitant decrease in pulmonary capillary stress failure. The administration of aerosolized nitric oxide donors and type V phosphodiesterase inhibitors is viewed as a novel therapeutic modality in the alleviation of capillary stress failure and EIPH in performance horses.

The foregoing and additional advantages and characterizing features of the present inventions will become clearly apparent upon reading of the ensuing detailed description together with the included drawing wherein:

### BRIEF DESCRIPTION OF THE FIGURES

FIG. 1 is a perspective view of a delivery system to provide an aerosolized compound to a horse.

FIG. 2 is a graphic representation of a type V phosphodiesterase inhibitors (“PDI”) effect on pulmonary artery pressure during maximum exercise in the equine.

FIG. 3 is a graphic representation of the effect of a type V PDI on systemic blood pressure during maximum exercise in the equine.

FIG. 4 is a graphic representation of the effect of a type V PDI on cardiac output during maximum exercise in the equine.

### DETAILED DESCRIPTION OF INVENTION

Nitric oxide (NO), previously known as endothelial derived relaxing factor, is a molecule that participates in the regulation of vascular tone. It is a selective pulmonary vasodilator and has been demonstrated to exhibit marked vasodilatory effects on the pulmonary circulation. Nitric oxide is an ideal local transcellular messenger because of its small size, lipophilic nature and short duration of action. In vascular endothelial cells, nitric oxide is synthesized from the terminal guanidine nitrogen of L-arginine and diffuses rapidly into subjacent vascular smooth muscle. There, the nitric oxide binds to the heme iron complex of soluble quanylate cyclase. The resulting nitrosyl-heme activates quanylate cyclase, stimulating the production of 3′5′-monophosphate (cGMP) and subsequent relaxation of vascular smooth muscle. When nitric oxide diffuses into the intravascular space, its biological activity is limited by avid binding to hemoglobin. In order for the therapeutics to be effective for the first embodiment of the present invention, the aerosolized nitric oxide donors need to be administered together with a type V phosphodiesterase inhibitor to the horse prior to the initiation of the high intensity exercise. And for the therapeutics to be effective for the second embodiment of the present invention, the type V phosphodiesterase inhibitor needs to be administered to the horse prior to the initiation of the high intensity exercise.

Similarly, cGMP needs to be sustained throughout the duration of the physical exercise to maintain the vascular smooth muscle in a relaxed state and thereby prevent pulmonary capillary stress failure. It is contemplated by the scope of the first embodiment of the present invention that continuous production of cGMP is provided by administration of aerosolized nitric oxide donors followed by an injection of a type V phosphodiesterase inhibitor just before exercise. Alternatively, for the second embodiment it is contemplated by the scope of the present invention that continuous production of cGMP is up regulated by administration of aerosolized type V PDI just before exercise.

### FIRST EMBODIMENT

The current invention is premised on the occurrence of pulmonary capillary stress failure resulting from excessively high transmural pulmonary artery pressure as the underlying mechanism leading to exercise induced pulmonary hemorrhage in the equine. Capillary stress failure produces occult hemorrhage into the lungs and tracheobronchial tree, which may be clinical (obvious bleeding through nares) or sub-clinical (endoscopic scoring, bronchial alveolar lavage sample, cytology) in its presentation. As a solution, the present invention introduces the concept of aerosolized nitric oxide donors in addition to an injection of type V phosphodiesterase inhibitors prior to exercise as a methodology for obtunding excessively high pulmonary hemodynamics and capillary stress failure and therefore EIPH in the equine. Examples of aerosolized nitric oxide donors are, and not limited to, FK 409(±)-(E)-4-ethyl-2-((E)-hydroxyimino)-s-nitro-3-hexenamide), SNAP (s-nitrosyl-acetylpenicillamine), DETANO (diethylenetriamine), NONOates (compounds from reacting NO with various nucleophiles) and nitricprusside.

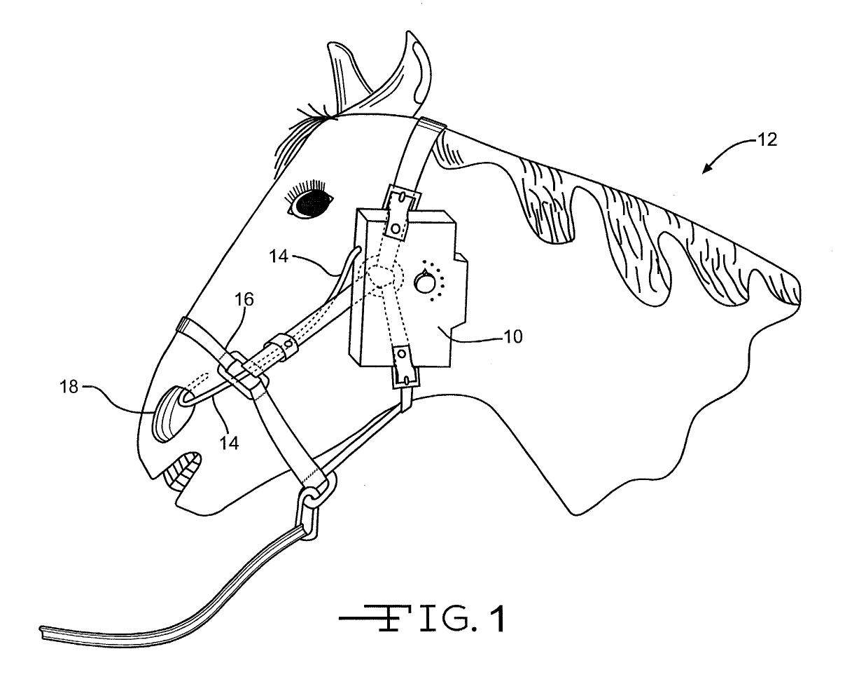

Turning now to FIG. 1, the aerosolization unit 10 is preferably anchored to the tack of the horse 12 in a surcingle near mid thorax. This aerosolization unit preferably quantifies the aerosolized NO donors to achieve the equivalent of 20-80 ppm of inhaled nitric oxide gas. Such a unit is available from Equatec Inc. located in Tofield, Alberta, Canada under the commercial name EPIH-NOX (II). A flexible conduit 14, for example, 0.25 I.D. flexible polytetrafluoroethylene tubing, connects to the EIPH-NOX (II) unit and traverses along the length of the dorsum of the neck and the bridle to the noseband 16 of the bridle. There it connects to the proximal end of a rigid, half-round flanged exit port 18, which is also connected to the noseband. The distal end of the half-round exit port is disposed proximate to the horse's nares. Typically, only one flanged exit port is required adjacent to either the right or the left nostril, depending on the trail of the flexible conduit 14, to deliver a physiological acceptable dose of aerosolized NO donors to satisfactorily decrease pulmonary artery pressure.

In use, the administration of aerosolized nitric oxide donors is a continuous flow of the gas throughout the horse's entire respiratory cycle during rest. The concentration of the gas delivered is dilutionally derived and therefore based on the individual's minute ventilation (or the quantity of air exchanged in a given minute). Thus the concentration of aerosolized nitric oxide donors delivered is a function of arbitrary flow rates.

Notwithstanding, the flow values outlined below in Table I illustrate a margin of error that allows for effective delivery of near optimal concentrations of the donors. With a source cylinder concentration of 50% aerosolized nitric oxide donors, the following serves as a guide for effective administration:

TABLE I

| | Target Aerosolized Nitric Oxide | Flow Meter Setting |
|---|---|---|
| Subject speed | Delivery (ppm) | on unit (l/min) |
| At rest | 20 | 300 ml/min |
| At rest | 50 | 790 ml/min |
| At rest | 80 | 1280 ml/min |

The present invention illustrates that improvements in nasal aerosolization are realized by, for example, gating the flow rate of the nitric oxide donor to a synchronous parameter of the animal such as the inspiratory phase of the respiratory cycle.

Synchronous inspiratory aerosolization is both advantageous and necessary in order to quantify aerosolized nitric oxide donor administration and minimize inadvertent human and/or mechanical error.

The use of aerosolized nitric oxide donors requires simultaneous administration of a type V phosphodiesterase inhibitor to be effectual in any treatment platform. There is a family of compounds known as phosphodiesterase inhibitors. These are enzymes that prevent catalyzing hydrolysis of an ester linkage by a phosphodiesterase. More specifically cGMP, which is the effectual compound at the cellular level and is derived from nitric oxide, is normally degraded very rapidly in-vivo by the action of endogenous phosphodiesterases. This reality would of consequence necessitate a constant instantaneous supply of aerosolized NO donors be maintained in order to accomplish the desired therapeutic effect. With the addition of a phosphodiesterase inhibitor coupled to aerosolized nitric oxide donors, there is a muting of the degradation of cGMP, resulting in a prolonged efficacy at the target level. Phosphodiesterase inhibitors useful with the present invention include, and not limited to, NA 1-(-6-chloro-4-(3,4-methylenedioxybenzyl)-aminoquinazolin-2-yl)-piperidine-4-carboxylate sesquihydrate, commercially available under the designation E4021. Other type V phosphodiesterase inhibitors include and are not limited to ONO 1505 which is ((4-[2-(2-hydroxyethoxy)ethylamino]-2-(1H-imidazol-1-yl)-6-methoxyquin azoline methanesulphonate)), DMPPO which is (1,3-dimethyl-6-(2-propoxy-5-methanesulfonylamidophenyl)pyrazol ol[3,4d]-pyrimidin-4-(5H)-one), Zaprinast which is 1,4-dihydro-5-(2-propoxyphenyl)-7H-1,2,2 triazolo-(4,5-d) pyrimidine-7-one), Sildenafil Citrate, Dipyridamole, and mixtures thereof.

The administration of aerosolized nitric oxide donors and a phosphodiesterase inhibitor embraces the following methodology. Prior to the initiation of the equine program (training/racing), the candidate is administered aerosolized nitric oxide donors via aerosolization at a fixed end concentration of 20-80 ppm and flow rate (predicated on the horse's tidal volume/minute ventilation) for a period of time ranging from about 2 to 10 minutes. Simultaneously, the phosphodiesterase inhibitor is given intravenously in a stringently pre-formulated dosing regimen. The aerosolized nitric oxide donor is withdrawn and the protocol terminated. This venue obviates the need for continuous application of the aerosolized nitric oxide donor during the equine's performance (training/racing) and facilitates the non-encumbrance of both the animal and the sophisticated techniques required with the employment of aerosolized nitric oxide donors alone.

The following examples describe the manner and process of nasal aerosolization of nitric oxide donors to an equine to reduce pulmonary artery pressure according to the present invention, and they set forth the best mode contemplated by the inventors of carrying out the invention, but they are not to be construed as limiting.

### EXAMPLE 1

All horses were studied at rest with measurement of baseline parameters, i.e., right atrial (RA), right ventricular (RV), pulmonary artery (PA), and pulmonary artery wedge pressure (PAW) for a minimum of 15 minutes. Also quantified were pulmonary capillary pressure (Pcap) (0.5 of average mean of PA and PAW pressure). After completion of these measurements, gradations of exercise intensity began on a high-speed treadmill. The horses walked for 1 minute at 2 m/sec. and continued in increments of 1 m/sec. in treadmill speed every minute until 6 m/sec. was achieved. Subsequent increases to 8 m/sec. for 1 minute, 10 m/sec. for 2 minutes and 13 m/sec. for 2 minutes followed. Immediately after the completion of 13 m/sec. treadmill exercise, catheter locations were confirmed and the microtip manometer signals checked against pressure signals from the fluid filled catheter. Data was collected 30 seconds to 1 minute prior to the end of each exercise level (i.e. 8, 10 and 13 m/sec.). Repeated measurements of RA, RV, PA, PAW and calculated Pcap were recorded. Following a rest period of 4 hours or more for the horse, the initiation of the aerosolized nitric oxide donor protocol begins.

The initial concentration of aerosolized nitric oxide donor to achieve 20 ppm of nitric oxide began with the horse at the treadmill speed of 8 m/sec. followed by 10 m/sec. and 13 m/sec. Since the administration of aerosolized nitric oxide donor is a constant flow system, the concentration of NO is dilutionally derived and therefore based on the animal's resting minute ventilation. For a minute ventilation concentration to achieve 20-80 ppm of nitric oxide, the horse needs to receive a pre-determined aerosolized concentration and therefore a derived fraction of the subject's VE (volume expired in one minute).

The rationale for the continuous flow of aerosolized nitric oxide donor is the assumed technical difficulty in gating the delivery of the aerosolized nitric oxide donor to the inspiratory phase of the horse's respiratory cycle. Hence, the aforementioned serves as a guide for implementing the administration of aerosolized nitric oxide donors. In an ideal environment, the exact minute ventilation of the animal is known. The required flow/concentration (i.e. 20, 50, 80 ppm of nitric oxide) is then easily calculated based on the minute ventilation.

Results of the tests using inhaled nitric oxide (gaseous form, not aerosolized) together with the type V phosphodiesterase inhibitor (E4021), as selective pulmonary vasodilators, have demonstrated a marked reduction in pulmonary vascular pressures and capillary stress failure and EIPH of greater than 30% by qualitative standards.

### SECOND EMBODIMENT

The second embodiment of the present invention is premised on the occurrence of pulmonary capillary stress failure resulting from excessively high transmural pulmonary artery pressure as the underlying mechanism leading to exercise induced pulmonary hemorrhage in the equine. Capillary stress failure produces occult hemorrhage into the lungs and tracheobronchial tree, which may be clinical (obvious bleeding through nares) or sub-clinical (endoscopic scoring, bronchial alveolar lavage sample, cytology) in its presentation. As a solution, the present invention introduces the concept of aerosolized type V phosphodiesterase inhibitors (PDI) prior to exercise as a methodology for obtunding excessively high pulmonary hemodynamics and capillary stress failure and therefore EIPH in the equine. Examples of aerosolized type V PDI are NA 1-(-6-chloro-4-(3,4-methylenedioxybenzyl)-aminoquinazolin-2-yl)-piperidine-4-carboxylate sesquihydrate, commercially available under the designation E4021. Other examples are ONO1505, DMPPO, Sildenafil Citrate, Dipyridamole, and Zaprinast, and mixtures thereof.

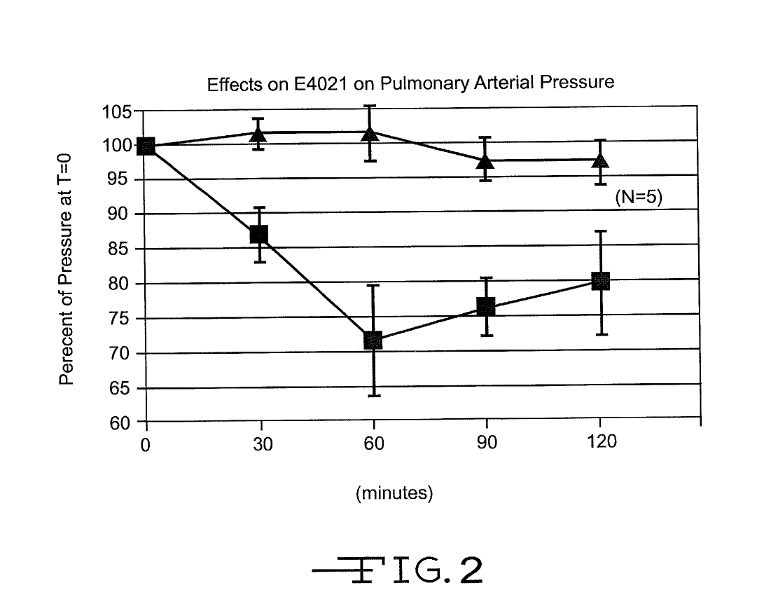

Returning to FIG. 1, the aerosolization unit 10 is preferably anchored to the tack of the horse 12 in a surcingle near mid thorax. This aerosolization unit preferably quantifies the aerosolized type V PDI to achieve the desired reduction in pulmonary artery pressure and hence capillary stress failure. Such a unit is available from Equatec Inc. Tofield, Alberta, Canada under the commercial name EPIH-NOX (II). The flexible conduit 14 connects to the EIPH-NOX (II) unit and traverses along the length of the dorsum of the neck and the bridle to the noseband 16 of the bridle. There it connects to the proximal end of a rigid, half-round flanged exit port 18, which is also connected to the noseband. The distal end of the half-round exit port is disposed proximate to the horse's nares. Typically, only one flanged exit port is required adjacent to either the right or the left nostril, depending on the trail of the flexible conduit 14, to deliver a physiological acceptable dose of aerosolized type V PDI to satisfactorily decrease pulmonary artery pressure. See FIG. 2.

As set forth in the first embodiment, the administration of aerosolized type V PDI is a continuous flow of the gas throughout the horse's entire respiratory cycle during rest. The concentration of the gas delivered is dilutionally derived and therefore based on the individual's minute ventilation (or the quantity of air exchanged in a given minute). Thus the concentration of aerosolized nitric oxide donors delivered is a function of arbitrary flow rates.

It is contemplated by the scope of the present invention that improvements in nasal aerosolization will be realized by, for example, gating the flow rate of the type V PDI to a synchronous parameter of the animal such as the inspiratory phase of the respiratory cycle. Synchronous inspiratory aerosolization is both advantageous and necessary in order to better quantify aerosolized PDI administration and minimize inadvertent human and/or mechanical error.

The family of compounds known as type V phosphodiesterase inhibitors are enzymes that prevent catalyzing hydrolysis of an ester linkage by a phosphodiesterase. More specifically cGMP, which is the effectual compound at the cellular level and is derived from nitric oxide, is normally degraded very rapidly in-vivo by the action of endogenous phosphodiesterases. There is evidence to support the application of type V PDI as stand alone agents in muting the degradation of cGMP, resulting in a significant reduction in pulmonary artery transmural pressure and hence pulmonary capillary stress failure during exercise. The administration of aerosolized type V phosphodiesterase inhibitor embraces the following methodology. Prior to the initiation of the equine program (training/racing), the candidate is administered aerosolized type V PDI at a fixed end concentration and flow rate (predicated on the horse's tidal volume/minute ventilation) for a period of time ranging from about 2 to 10 minutes. The following example describes the manner and process of nasal aerosolization of a PDI to an equine to reduce pulmonary artery pressure according to the present invention, and they set forth the best mode contemplated by the inventors of carrying out the invention, but they are not to be construed as limiting.

### EXAMPLE 2

All horses were studied at rest with measurement of baseline parameters i.e. right atrial (RA), right ventricular (RV), pulmonary artery (PA), and pulmonary artery wedge pressure (PAW) for a minimum of 15 minutes. Also quantified were pulmonary capillary pressure (Pcap) (0.5 of average mean of PA and PAW pressure). After completion of these measurements, gradations of exercise intensity began on a high-speed treadmill. The horses walked for 1 minute at 2 m/sec. and continued in increments of 1 m/sec. in treadmill speed every minute until 6 m/sec. was achieved. Subsequent increases to 8 m/sec. for 1 minute, 10 m/sec. for 2 minutes and 13 m/sec. for 2 minutes followed. Immediately after the completion of 13 m/sec. treadmill exercise, catheter locations were confirmed and the microtip manometer signals checked against pressure signals from the fluid filled catheter. Data was collected 30 seconds to 1 minute prior to the end of each exercise level (i.e. 8, 10 and 13 m/sec.). Repeated measurements of RA, RV, PA, PAW and calculated Pcap were recorded. Following a rest period of 4 hours or more for the horse, the initiation of the aerosolized type V PDI protocol begins.

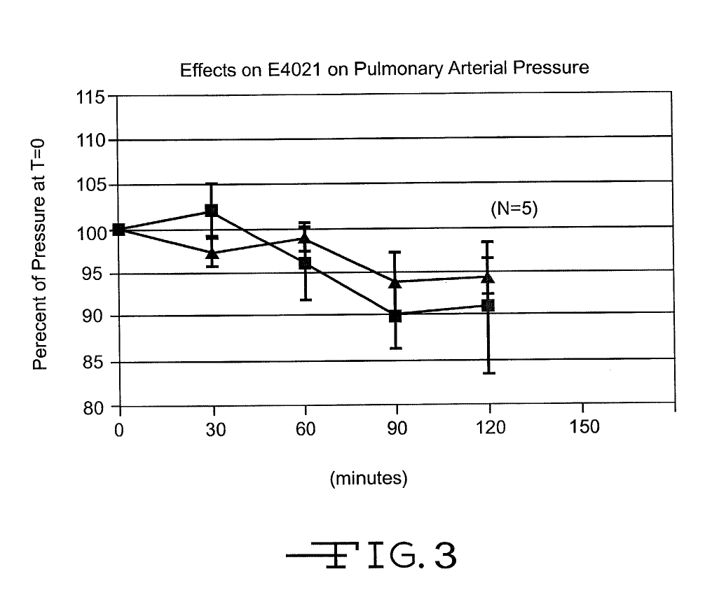

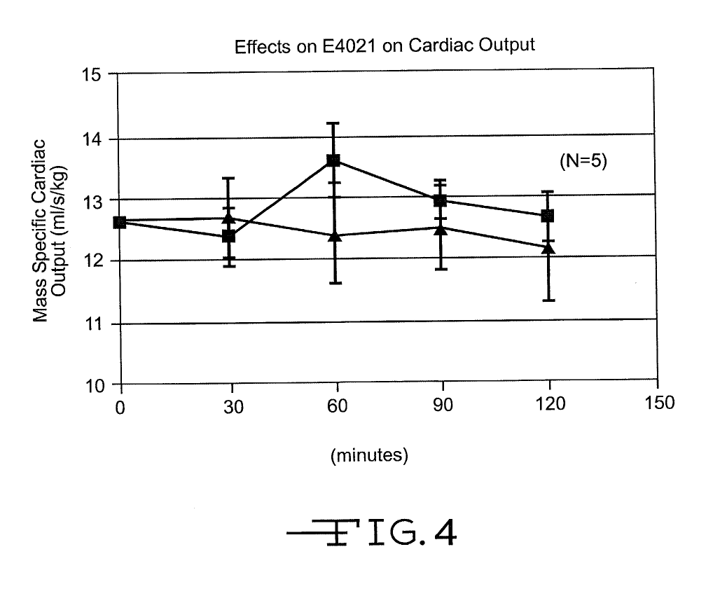

The rationale for the continuous flow of aerosolized type V PDI is the assumed technical difficulty in gating the delivery of the aerosolized PDI to the inspiratory phase of the horse's respiratory cycle. In an ideal environment, the exact minute ventilation of the animal is known. The required flow/concentration is then easily calculated based on the minute ventilation. Results of the tests using the type V phosphodiesterase inhibitor E4021, as a selective pulmonary vasodilator, have demonstrated a marked reduction in pulmonary vascular pressures and capillary stress failure with no untoward systemic effects. See FIGS. 2, 3, and 4.

While the present invention has been particularly described in connection with certain specific embodiments thereof, it is to be understood that this is by way of illustration and not limitation, and the scope of the appended claims should be construed as broadly as the prior art will permit.

We claim:

1. A method of preventing exercised induced pulmonary hemorrhage in a horse, comprising the steps of:
aerosolizing a nitric oxide donor;
combining the aerosolized nitric oxide donor with a simultaneous injection of a type V phosphodiesterase inhibitor;
exercising the horse; and
administering a physiological acceptable quantity of the aerosolized nitric oxide donor into the horse's lungs to obtund pulmonary artery pressure to thereby reduce pulmonary capillary stress failure into the lungs to a degree sufficient to prevent hemorrhaging.

2. The method of claim 1 further comprising regulating the quantity of aerosolized nitric oxide donors delivered to the lungs dependent on the horse's level of exercise.

3. The method of claim 1 further comprising regulating the aerosolized flow rate of the nitric oxide donor to a synchronous parameter of the horse's inspiratory phase of the respiratory cycle.

4. The method of claim 1 further comprising providing an aerosolized nitric oxide donor comprising the nitric oxide resulting in a concentration level equivalent to inhaled gaseous nitric oxide of about 20 to 80 ppm.

5. A method of stimulating the production of cyclic 3′,5′-monophosphate in the lungs of an equine to prevent exercised induced pulmonary hemorrhage, comprising the steps of:
aerosolizing a nitric oxide donor;
combining the aerosolized nitric oxide donor with a simultaneous injection of a type V phosphodiesterase inhibitor;
exercising the horse; and
administering a physiological acceptable quantity of a nitric oxide donor into the horse's lungs to stimulate cyclic 3′,5′-monophosphate production prior to initiation of the exercise to thereby reduce pulmonary capillary stress failure in the horse's lungs to a degree sufficient to prevent hemorrhaging.

6. The method of claim 5 further comprising preemptive administration of an aerosolized nitric oxide donor prior to the onset of the exercise.

7. The method of claim 5 further comprising regulating the flow rate of the aerosolized nitric oxide donor to a synchronous parameter of the horse's inspiratory phase of the respiratory rate.

8. The method of claim 5 further comprising providing an aerosolized nitric oxide donor resulting in the equivalent inhaled nitric oxide at a level of about 20 to 80 ppm.

9. A method of preventing exercised induced pulmonary hemorrhage in equine, comprising the steps of:
aerosolizing a nitric oxide donor;
administering a physiological acceptable quantity of the aerosolized nitric oxide donor into the horse's lungs to obtund pulmonary artery pressure based on the anticipated duration and magnitude of exercise to which the horse is expected to be immediately subjected and intravenously administering a phosphodiesterase inhibitor to the horse.
exercising the horse.

10. The method of claim 9 wherein the phosphodiesterase inhibitor is NA 1-(-6-chloro-4-(3,4-methylenedioxybenzyl)-aminoquinazolin-2-yl)-piperidine-4-carboxylate sesquihydrate, ONO 1505 which is ((4-[2-(2-hydroxyethoxy)ethylamino]-2-(1H-imidazol-1-yl)-6-methoxyquin azoline methanesulphonate)), DMPPO which is (1,3-dimethyl-6-(2-propoxy-5-methanesulfonylamidophenyl)pyrazol ol[3,4d]-pyrimidin-4-(5H)-one), Zaprinast which is 1,4-dihydro-5-(2-propoxyphenyl)-7H-1,2,2 triazolo-(4,5-d) pyrimidine-7-one), Sildenafil Citrate, Dipyridamole, and mixtures thereof.

11. The method of claim 9 further comprising administering the phosphodiesterase inhibitor in a dosage of about 1 to 100 ig/kg.

12. The method of claim 9 further providing the aerosolized nitric oxide donor resulting in a level equivalent to inhaled nitric oxide of about 20 to 80 ppm.

13. A method of stimulating the production of cyclic 3′,5′-monophosphate in the lungs of a horse to prevent exercise induced pulmonary hemorrhage, comprising the steps of:
providing an aerosolized gas comprising nitric oxide donor;
administering a physiological acceptable quantity of the aerosolized gas into the horse's lungs in a quantity sufficient to produce cyclic 3′,5′-monophosphate at a quantity sufficient to prevent hemorrhaging based on the anticipated duration and magnitude of exercise to which the horse is expected to be immediately subjected;
intravenously administering a phosphodiesterase inhibitor to the horse in a quantity sufficient to mute the degradation of 3′,5′-monophosphate throughout the anticipated duration of exercise;
discontinuing the aerosolization; and
exercising the horse.

14. The method of claim 13 wherein the phosphodiesterase inhibitor is selected from the group consisting of NA 1-(-6-chloro-4-(3,4-methylenedioxybenzyl)-aminoquinazolin-2-yl)-piperidine-4-carboxylate sesquihydrate, ONO 1505 which is ((4-[2-(2-hydroxyethoxy)ethylamino]-2-(1H-imidazol-1-yl)-6-methoxyquin azoline methanesulphonate)), DMPPO which is (1,3-dimethyl-6-(2-propoxy-5-methanesulfonylamidophenyl)pyrazol ol[3,4d]-pyrimidin-4-(5H)-one), Zaprinast which is 1,4-dihydro-5-(2-propoxyphenyl)-7H-1,2,2 triazolo-(4,5-d) pyrimidine-7-one), Sildenafil Citrate, Dipyridamole, and mixtures thereof.

15. The method of claim 13 further comprising administering the phosphodiesterase inhibitor in a dosage of about 1 to 100 μ/kg.

16. The method of claim 13 further comprising providing the aerosolized gas comprising the nitric oxide donor resulting in an equivalent inhaled nitric oxide level of about 20 to 80 ppm.

17. An apparatus for preventing exercised induced pulmonary hemorrhage in a horse, which comprises:
a container adapted to be carried by the horse prior to exercise, wherein the container has an aerosolized gas comprising a nitric oxide donor and a nozzle;
the nozzle is in fluid flow communication with the container, wherein the nozzle is near proximate to at least one nostril of the horse to transmit the aerosolized gas of a physiological acceptable quantity to the horse to reduce pulmonary capillary stress failure in the lungs to a degree sufficient to prevent hemorrhaging.

18. The apparatus of claim 17 further including a self contained aerosolization/nebulization unit.

19. The apparatus of claim 17 wherein the aerosolized gas includes the nitric oxide donor at a concentration equivalent to an inhaled nitric oxide about 20 to 80 ppm.

20. A method of preventing exercised induced pulmonary hemorrhage in the equine, comprising the steps of:
aerosolizing a type V phosphodiesterase inhibitor;
exercising the horse; and
adminstrating a physiological acceptable quantity of the aerosolized type V phosphodiesterase inhibitor into the horse's lungs to obtund pulmonary artery pressure to thereby reduce pulmonary capillary stress failure into the lungs to a degree sufficient to prevent hemorrhaging.

21. The method of claim 20 further comprising regulating the quantity of aerosolized type V phosphodiesterase inhibitor delivered to the lungs dependent on the horse's level of exercise.

22. The method of claim 20 further comprising regulating the aerosolized flow rate of the type V phosphodiesterase inhibitor to a synchronous parameter of the horse's inspiratory phase of the respiratory cycle.

23. The method of claim 20 further comprising providing the aerosolized type V phosphodiesterase inhibitor to up regulate endogenous nitric oxide levels.

24. A method of stimulating the production of cyclic 3′,5′-monophosphate in the lungs of a horse to prevent exercised induced pulmonary hemorrhage, comprising the steps of:
aerosolizing a type V phosphodiesterase inhibitor;
exercising the horse; and
adminstrating a physiological acceptable quantity of the aerosolized type V phosphodiesterase inhibitor into the horse's lungs to stimulate cyclic 3′,5′-monophosphate production prior to initiation of the exercise to thereby reduce pulmonary capillary stress failure in the lungs to a degree sufficient to prevent hemorrhaging.

25. The method of claim 24 further comprising preemptive administration of a aerosolized type V phosphodiesterase prior to the onset of the exercise.

26. The method of claim 24 further comprising regulating the flow rate of the aerosolized type V phosphodiesterase to a synchronous parameter of the horse's inspiratory phase of the respiratory cycle.

27. The method of claim 24 further comprising providing the aerosolized type V phosphodiesterase resulting in an up regulation of the endogenous nitric oxide level.
